(19) United States
(12) Patent Application Publication
(10) Pub. No.: US 2018/0169177 A1
(43) Pub. Date: Jun. 21, 2018

(54) POLYPEPTIDE SPECIFICALLY BINDING TO CD34 MOLECULE AND USE THEREOF

(71) Applicants: Guangzhou Yidai Pharmaceutical Co., Ltd., Guangzhou City, Guangdong (CN); Sun Yat-sen University, Guangzhou, Guangdong (CN)

(72) Inventors: HUA WANG, Guangzhou City (CN); LONG ZHAO, Guangzhou City (CN); HAIXIA ZHANG, Guangzhou City (CN); YAN ZHANG, Guangzhou City (CN)

(21) Appl. No.: 15/119,418

(22) PCT Filed: Sep. 22, 2015
(86) PCT No.: PCT/CN2015/090211

§ 371 Date: Aug. 17, 2016

(30) Foreign Application Priority Data
Aug. 18, 2015 (CN) 201510507116.9

(51) Int. Cl. A61K 38/04; G01N 33/68; G01N 33/574; G01N 33/50; G01N 33/543

(52) U.S. Cl. CPC A61K 38/04; G01N 33/68; G01N 33/57426; A61K 2035/124; G01N 33/5002; G01N 33/54346; G01N 33/5434; G01N 33/5008

(57) ABSTRACT

A polypeptide is provided specifically binding to CD34 molecule and use thereof, the polypeptide is selected from at least one of polypeptide 17 and polypeptide 19, amino acid sequences of the polypeptide 17 and the polypeptide 19 are as shown in SEQ ID NO: 1 and SEQ ID NO: 2 in sequence listing. The polypeptide provided by the present application can specifically bind to CD34, and can be produced by artificial synthesis or genetic engineering method. Compared with antibody, the polypeptide provided by the present invention has characteristics such as low molecular weight, easy preparation and less immunological rejection; and it has little toxic side-effects, its binding to the CD34+ cell won't obviously kill the target cell and inhibit its proliferation. The polypeptide provided by the present invention can be used as markers for CD34 positive expression cells, and also can be used as substitute of immunofluorescence CD34 antibody.

### TECHNICAL FIELD

The present invention relates to the field of biological medicine, and particularly to a polypeptide specifically binding to CD34 molecule and use thereof.

### BACKGROUND

CD34 molecule is an advanced glycosylated type-I transmembrane protein with a molecular weight of 105 to 120 kD and a particular structure, and it is specifically expressed on the surfaces of hemopoietic stem/progenitor cells (HSC/HPC) of human beings and other mammals. The expression quantity of CD34 will decrease gradually with the maturity of hematopoietic cells. Besides, CD34 molecule is also expressed in normal and tumorous microvascular endothelial cells, but its roles of regulation in the vascular endothelium is not clear yet.

It has been shown in the previous research that CD34 molecule plays an important part in the adhesion between mediating cells, and it involves in the transport, localization and homing of the HSC/HPC. The binding between the CD34 molecules and the marrow mesenchyme layer will be enhanced if the CD 34 monoclonal antibodies bind to the extracellular domain of the CD34 molecules. During the homing of HSC/HPC, CD34 molecules on the cell surfaces will firstly bind to L- selectin of the endothelial cells and marrow stroma to start the adhesion, then they will pass through the endothelium layer, localize in the marrow extravascular stroma, proliferate and differentiate, and then complete the homing process. Adhesion signal mediated by CD34 regulates the expression of G-protein-coupled receptors, such as CXCR4, through tyrosine protein kinase signal path, so as to facilitate the chemotaxis, adhesion and migration of cells. Tyrosine protein kinase inhibitor and CXCR4 blocker can be used to inhibit the adhesion of cells, and activate HSC/HPC and make them enter the peripheral blood circulation.

Regulation drugs for the adhesion of HSC/HPC are widely used to repair the myelosuppression. In the therapeutic process of transplantation of HSC/HPC, the drugs for activating HSC/HPC are firstly applied to inhibit the adhesion of cells, making the HSC/HPC easily pass through the medullary blood barrier and enter the peripheral blood circulation, which is convenient for enriching and adopting the HSC/HPC; after the transplantation, drugs for promoting the adhesion of HSC/HPC are used to enhance the adhesion of CD34, which improves the binding between the CD34 molecules and surface molecules of marrow stroma cells, and promotes the colonization, homing, recovery and reconstruction of hematopoiesis and immune function of HSC/HPC.

Clinically, drugs for activating HSC/HPC are also used to inhibit the adhesion of cells, making HSC/HPC enter the peripheral blood circulation to repair and cure tissue damages, such as cardio-cerebral infraction, etc.

In the clinical application, the detection of CD34 molecules may be used to confirm, count, enrich and purify the HSC/HPC, and it is significant in the transplantation of HSC and tumor biotherapy. Using CD34 to purify the HSC/HPC may decrease the amount of T - cells in graft during the transplantation of allogenetic hematopoietic stem cells, whereby the acute and chronic graft-vs-host disease (GVHD) caused by T - cells will be effectively prevented. In addition, CD34 molecules may be used for the diagnosis and classification of leukemia, and CD34 molecules are specifically expressed in some undifferentiated or poorly differentiated leukemia cells. For example, the CD34 expression differences in acute myeloid leukemia (AML) has a correlation with clinical chemotherapeutic effect and prognosis. Furthermore CD34 molecule is considered as the most sensitive blood vessel endothelium marker, and it can be used to count the capillaries, and determine the condition of tumor angiogenesis and curative effect of tumor angiogenesis inhibitors.

CD34 also involves in inflammatory response and homing of lymphocyte, and CD34 antibody is an indispensable molecular probe for the in-depth researches in aspects such as mechanisms of CD34 molecule in hematopoiesis, inflammatory response and angiogenesis, as well as separation and purification of HSP/HPC, and diagnose of acute myelogenous leukemia. So far only a few countries, such as U.S.A, Britain, France, Denmark, have developed about 10 mouse anti-human CD34 antibodies through classic hybridoma technique. All of these CD34 antibodies specifically bind to CD34 molecule, and could recognize HSC/HPC, vascular endothelial cells and some leukemia cells. However there are still some problems in the preparation and clinic application of CD34 antibodies: firstly CD34 molecule has weak immunogenicity, so it is difficult to stimulate organism to produce B-cells capable of secreting specific antibodies, which will lead to large workload and low success rate of preparation of CD34 antibodies, whereby the cost is high and production cycle is long; secondly, during the transplantation of HSC, these mouse-derived antibodies will enter human body along with HSC inevitably, which will cause human anti-mouse antibody response (HAMA), and leave a medical safety risk.

### SUMMARY OF THE INVENTION

In order to overcome the drawbacks in the prior art, the present application provides a polypeptide which specifically binds to CD34 molecule, with characteristics of easy preparation, low molecular weight, and less immunological rejection.

In order to achieve the above object, the following technical solutions are provided: providing a polypeptide specifically binding to CD34 molecule, wherein the polypeptide is selected from at least one of polypeptide 17 and polypeptide 19, amino acid sequences of the polypeptide 17 and the polypeptide 19 are as shown in SEQ ID NO: 1 and SEQ ID NO: 2 in the sequence listing respectively.

The polypeptide provided in the present invention can specifically bind to CD34 molecules, and can be produced by the method of artificial synthesis or genetic engineering. The polypeptide also can be called as a non-antibody binding protein, wherein the term of non-antibody binding protein is called relative to the characteristics of specific binding of antibody/antigen, and the polypeptide further can be called as a scaffolds binding protein. In three dimensional structure, such protein can bind to specific molecules through scaffolds formed by amino acids on its surface. Compared with the antibody, the protein has characteristics of low molecular weight, strong permeability, heat resistance and good stability, and it is easy to control and suitable for large-scale production.

The polypeptide provided in the present invention can bind to CD34⁺ HSC, vascular endothelia cell and leukemia cell (particularly such as K562 leukemia cell), and cannot bind to CD34⁻ MNNG/HOS.

The polypeptide provided in the present invention will not affect the proliferation rate of CD34⁺ cell after binding to it.

The polypeptide provided in the present invention may be used as a marker of CD34 positive expression cells, particularly such as markers of ductal epithelia cells and vascular endothelia cells.

The polypeptide provided in the present invention may also be used for the identification, enriching, screening or purification of stem cells, particularly HSC/HPC.

The polypeptide provided in the present invention may also be used to prepare reagents for diagnosis or typing of leukemia.

The polypeptide provided in the present invention may also be used to prepare CD34 target drugs, the active components of which comprise the polypeptide mentioned above.

The polypeptide provided in the present invention may be used to regulate the adhesion and homing of CD34 positive cells, for example, it could be used to prepare pharmaceutic formulations capable of inhibiting the adhesion and homing of CD34 positive cells.

The polypeptide provided in the present invention may also be used to prepare formulations for adsorbing and enriching CD34 positive cells, for example, it may be used in tissue regeneration area for adsorbing HSC, and promoting regeneration and healing of the tissue; and it is used for the preliminary screening of cancel cells of leukemia patients.

When the polypeptide of the present invention is used to prepare drugs or formulations as mentioned above, the drugs or formulations may also include pharmaceutically acceptable carriers, excipients, and the like. These carriers or excipients can be selected according to dosage form to be prepared by one skilled in the art. The carriers, for example, may be one or more of diluent, excipient, adhesive, wetting agent, disintegrant, sorbefacient, surfactant, adsorbing carrier, lubricant, etc.; the excipients, for example, may include flavoring agent, sweetening agent, etc. The dosage form of drugs, for example, may be capsule, soft capsule, tablet, oral liquid, dispersible tablet, freeze-dried powder injection, injection or dropping pill, etc.

The polypeptide provided in the present invention may also be used as drug targeting molecule, which can be used to prepare drug targeting formulations, for example, the drug targeting formulations oriented to CD34⁺ cells.

The polypeptide provided in the present application may also be used to separate or detect CD34 positive cells, in place of the fact that CD34 antibody is used to separate or detect CD34 positive cells in the prior art.

The polypeptide provided in the present invention meets the requirements of following medical applications and medical researches:

1) it can be used to detect, enrich and purify HSC/HPC;

2) it can be used as a blood vessel endothelium marker;

3) it can be used for diagnosis and typing of leukemia;

4) it can be used as pharmaceutical targeting molecule;

5) it can be used as a substitute of immunofluorescent CD34 antibody;

6) it can be used to inhibit the adhesion of CD34 positive cells; and

7) it can be used to adsorb stem cells, and promote tissue regeneration and repair.

Besides, the polypeptide provided in the present invention also has following advantages:

1) the polypeptide has high binding efficiency, and it can specifically bind to CD34⁺ stem cell, HSC, vascular endothelial cells, and K562 leukemia cell;

2) the polypeptide has little toxic side-effects, binding to the CD34⁺ cell won't obviously kill the target cell and inhibit its proliferation;

3) the polypeptide has characteristics such as low molecular weight, good permeability, poor degeneration ability and low immunogenicity;

4) the polypeptide is easy to prepare with low lost, and it not only can be synthesized directly by artificial synthesis method, but also can be prepared by genetic engineering method, which avoids hidden danger of foreign proteins such as those produced by antibody animals, this enhances the medical safety of separation and preparation of stem cells, and it is conducive to less complication of stem cell transplantation.

### BRIEF DESCRIPTION OF THE DRAWINGS

FIG. 1 shows the PCR results of monoclonal phages 17, 19 and 24, these monoclonal phages, screened by phage display technology, are capable of binding to CD34 molecules. Except that the first band indicates Mark, there are PCR bands of phages 17, 19 and 24 successively from left to right.

FIGS. 2a to 2b show the results of fluorescence intensity of CD 34 fluorescent polypeptide binding CD34 molecules after a precipitation of CD34 antibodies, detected by fluorospectro photometer, wherein the CD34 molecules of K563 leukemia cells are performed co-immunoprecipitation with CD34 antibodies, then are bound to the polypeptide; wherein FIG. 2a shows testing results of the binding between polypeptide 17 and CD34 molecule; FIG. 2b shows testing results of the binding between polypeptide 19 and CD34 molecule.

FIGS. 3a to 3b show immunofluorescence photo illustrating the binding (red fluorescence) between CD34 molecules in K562 leukemia cells and CD34 antibodies and the binding (green fluorescence) between CD 34 molecules in K562 leukemia cells and CD34 binding polypeptide; wherein FIG. 3a is an immunofluorescence photo (1000×) illustrating the binding between polypeptide 17 and CD34 molecule in K562 cells; FIG. 3b is an immunofluorescence photo (1000×) illustrating the binding between polypeptide 19 and CD34 molecule in K562 cells.

FIG. 4 shows the experimental results of adhesion between CD34 binding polypeptide and CD34 positive cells.

FIG. 5 is a schematic diagram of magnetic separation.

FIG. 6a is an immunofluorescence photo showing the binding between CD34 binding polypeptide and ductal epithelium cells and vascular endothelia cells in section of human salivary gland tissue, wherein green fluorescence is indicative of the binding site with polypeptide, and blue fluorescence is indicative of DAPI nucleus staining.

FIG. 6b shows negative control of CD34 binding polypeptide, wherein blue fluorescence is indicative of DAPI nucleus staining.

FIG. 6c is an immunofluorescence photo of CD34 expression in section of human salivary gland tissue, wherein red fluorescence is indicative of CD34 expression, and blue fluorescence is indicative of DAPI nucleus staining.

FIG. 6d shows the H&E staining result of section of human salivary gland tissue.

FIG. 7a is a microphotograph of MNNG/HOS in bright field; and FIG. 7b is a fluorescence microscope photo showing the binding between cells in that area and polypeptide 19.

FIG. 8a is a microphotograph of MNNG/HOS in bright field; and FIG. 8b is a fluorescence microscope photo showing the binding between cells in that area and polypeptide 17.

FIG. 9 shows proliferation curves of K562 cells, after adding CD34 binding polypeptides 17 and 19 respectively into a low-serum culture solution of K562 cells.

### DETAILED DESCRIPTION OF PREFERRED EMBODIMENTS

The technical solutions of the present invention are further explained with reference to accompanying figures.

Unless specified otherwise, the term “percentage” refers to mass percent herein; unless specified otherwise, reagents used herein are commercially available.

### EXAMPLE 1

### Screening of CD34 Binding Phage

(1) Enriching CD34 Binding Phages by Taking CD34 as the Target Molecule

Use 5 μg/ml CD34 molecules (purchased from PeproTech) to coat a 96-hole plate and seal it with 5% skim milk powder, mix T7 phage lysate displaying random polypeptide in human liver cancer cDNA library (Novagen) with 3% skim milk powder at a mass ratio of 1:1, incubate the 96-hole plate coated with CD34 molecules for 30 minutes. Elute and remove the nonspecific binding phage with flushing fluid, enrich CD34 binding T7 phage, repeat the screening until the phage library recovery is not increased. The culture dish not being coated with CD34 molecules but sealed by 5% skim milk powder is taken as a control group.

The titer of phage added in the first screening round is 6.4×10¹⁰ cfu, and the titer of recycled phage is 5.4×10⁵ cfu. Amplify the recycled phage to reach a titer of 4.2×10⁹ cfu and reuse it in the second screening round, the titer of recycled phage in the second screening round is 5.8×10² cfu. Likewise, amplify the screened phage to reach a titer of 8.53×10⁹ cfu and reuse it to continue the third screening round, the titer of recycled phage is 1.62×10⁴ cfu. After calculation, the screening recovery didn't increase since the first screening round (as shown in Table 1). Preserve the phage library after screening.

TABLE 1
Screening result of phages

| Screen- | Experimental group | | Control group | |
|---|---|---|---|---|
| ing | Added | Recycled | Added | Recycled |
| round | phage | phage | phage | phage |
| Round | 6.4 × 10¹⁰ cfu | 5.4 × 10⁵ cfu | 1.74 × 10⁵ cfu | 8.43 × 10⁻⁶ |
| 1 | | | | |
| Round | 4.2 × 10⁹ cfu | 5.8 × 10² cfu | 4.2 × 10² cfu | 1.38 × 10⁻⁷ |
| 2 | | | | |
| Round | 8.53 × 10⁹ cfu | 1.62 × 10⁴ cfu | 1.60 × 10⁴ cfu | 1.70 × 10⁻⁶ |
| 3 | | | | |

### (2) Screening of CD34 Binding Monoclonal Phages

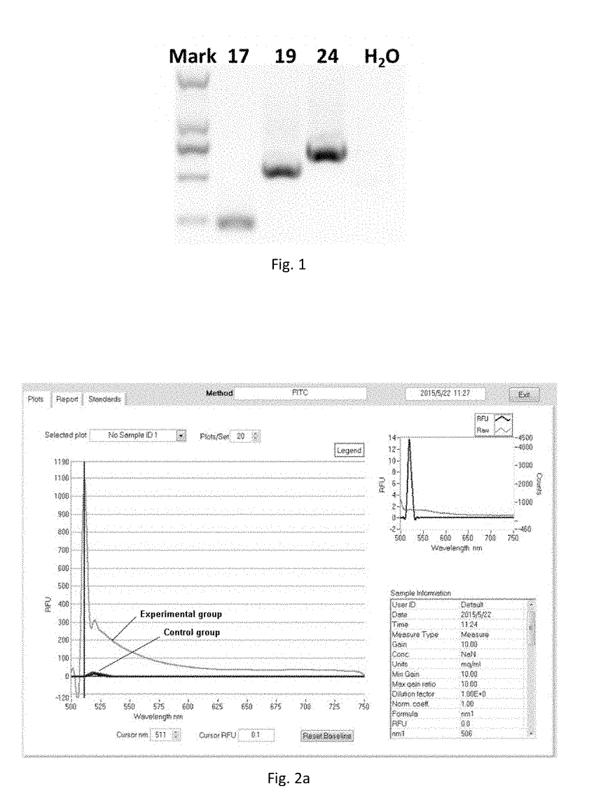

Select 80 monoclonal phages randomly in the enriched phage library to amplify, and then bind them to a 96-hole plate coated with CD34. Elute and remove the nonspecific binding monoclonal phages, and test the binding efficiency of each monoclonal phage through spectrophotometer. Select three CD34 binding monoclonal phages with high binding efficiency, and name them as monoclonal phages 17, 19 and 24 respectively according to the selection sequence of monoclonal phages. Perform bacterial liquid PCR for these three monoclonal phages, and the results are shown in FIG. 1, wherein each bacterial liquid PCR band of phages 17, 19 and 24 is single band, which proves that they are monoclonal phages.

(3) Sequencing and Synthesis of Polypeptides

Send three monoclonal phages to Guangzhou Ige Biotechnology Ltd. to sequence the cDNA sequence inserted into the CD34 molecule binding positive monoclonal phage, analyze and obtain the amino acid sequence specifically binding to CD34 molecule. The displayed amino acid sequences of phages 17 and 19 are as shown in SEQ ID NO: 1 and SEQ ID NO: 2 in the sequence listing. The displayed amino acid sequence of phage 24 has still been scientifically verifying yet, so it is not shown herein.

The displayed polypeptides of synthetic phages 17 and 19 (synthesized by China Peptides Co., Ltd.) are named as polypeptide 17 and polypeptide 19, the amino acid sequences of which are as shown in SEQ ID NO: 1 and SEQ ID NO: 2 in the sequence listing, a FITC (green fluorescence) marker is added to the N-end, with purity >95%.

### EXAMPLE 2

### Co-Immunoprecipitation of CD34 Binding Polypeptide

(1) extracting total protein: wash K562 cell once with pre-cooled PBS buffer solution, remove the PBS, add 500 μL non-denatured protein lysate containing protease inhibitor into the culture dish, collect cells into a 1.5 mL centrifuge tube, perform ultra sonication for 4 times, 4 seconds each time, leave them on the ice for 30 minutes, and centrifuge them at 8000 rpm at 4° C. for 20 minutes, transfer the supernatant into a new centrifuge tube to obtain total protein products;

(2) pretreatment of cellular total protein: add 50 μL confining liquid of normal goat serum into the cellular total protein products, and incubate them on the ice for 1 hour, then add 100 μL G-protein microsphere suspension (Calbiochem), incubate them at 4° C. for 20 minutes, centrifuge and remove the G-protein microsphere.

(3) Co- immunoprecipitation: add CD34 antibodies (AR) into the total protein pretreated in step (2), take it as experimental groups, set control groups, wherein equal amount of PBS antibody diluent is added, and incubate them at 4° C. for 8 hours;

(4) Binding of polypeptides and purification of G-protein antibodies: add polypeptides 17 and 19 into respective experimental groups and control group respectively, meanwhile add G-protein microsphere suspension, and incubate them at 4° C. for 4 hours;

(5) Washing and elution: centrifuge at 4° C. to remove the supernatant, use washing liquor to wash for three times, centrifuge and remove the supernatant, add 2× SDS buffer solution in a volume the same as that of G-protein microsphere, treat them at 50° C. for 10 minutes, centrifuge and remove the microspheres, transfer the supernatant into a new EP tube, and add DTT (dithiothreitol) until the final concentration is 100 mM;

(6) Testing fluorescence intensities of experimental groups and control groups using micro fluorospectro photometer (NanoDrop 3300).

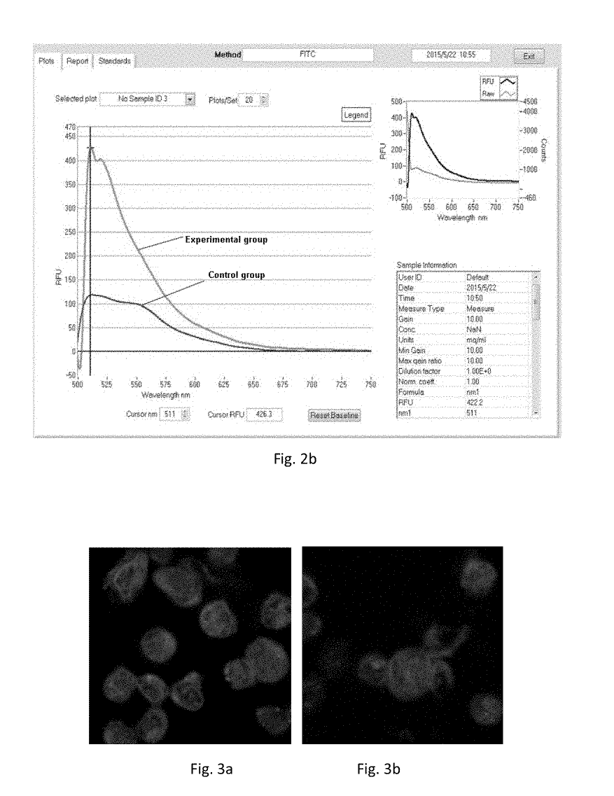

As shown in FIGS. 2a and 2b (FIG. 2a shows fluorophotometer testing result of polypeptide 17, FIG. 2b shows fluorophotometer testing result of polypeptide 19), it could be seen from FIGS. 2a and 2b, protein after the co-immunoprecipitation with CD34 antibodies can specifically bind to polypeptides 17 and 19, and the polypeptide 19 has a better binding specificity than the polypeptide 17.

### EXAMPLE 3

### Binding Between CD34 Binding Polypeptide and CD34 Molecules of CD34 Positive Cells

(1) Preparation of cells: incubate K562 cell suspension in an incubator (37° C., 5% CO₂). When the cell density reaches 80%, centrifuge and remove the culture solution, wash it once using PBS buffer solution, centrifuge it, take the cell suspension and drop it onto a glass slide coated with polylysine, and use cold acetone to fix for 10 minutes after drying.

(2) Use PBS buffer solution to wash for three times, 5 minutes each time, and then use 5% BSA confining liquid to seal it at room temperature for 1 hour.

(3) Incubation of primary antibodies: drop the primary antibody diluent of CD34 antibodies (AR) onto a slide, incubate it at 4° C. over night. Use PBS buffer solution to wash it for three times, 5 minutes each time.

(4) Incubation of second antibodies and binding to polypeptide: add polypeptides 17 and 19 into diluted anti-rabbit antibodies labeled by Alexa Fluor 561 respectively, drop them onto cover glasses, incubate them at room temperature and keep shielded from light for 1 hour. Use PBS buffer solution to wash for three times, 5 minutes each time.

(5) Use 1 μg/mL DAPI to stain at room temperature and keep shielded from light for 5 minutes, use PBS buffer solution to wash it for three times, 5 minutes each time.

(6) After mounting with anti-fluorescence quenching agent, use laser scanning confocal microscope to observe and photograph.

The results are shown in FIGS. 3a to 3b, wherein red fluorescence is indicative of the location labeled by CD34 antibodies, green fluorescence is indicative of the location labeled by CD34 binding polypeptides (FIG. 3a shows the polypeptide 17, and FIG. 3b shows the polypeptide 19), blue is indicative of DAPI nucleus staining, the red fluorescence and the green fluorescence overlap with each other, which proves that the polypeptides 17 and 19 have bound to CD34 molecules.

As can be seen from the experimental results of the example, the polypeptides 17 and 19 may be used in replace of CD 34 antibodies in the separation or detection of CD34 positive cells, such as in the separation of stem cells, diagnosis or typing of leukemia. They also can be used in the development of reagent or materials for adsorbing CD34 positive cells.

The experimental results of the example also show the advantages of replacing antibodies with the polypeptides 17 and 19, the polypeptides being labeled by fluorescence can bind to molecules on cellar surfaces directly within a short response time, which is convenient for clinical application.

### EXAMPLE 4

### Effect of CD34 Binding Polypeptide on Adhesion of CD34 Positive Cells

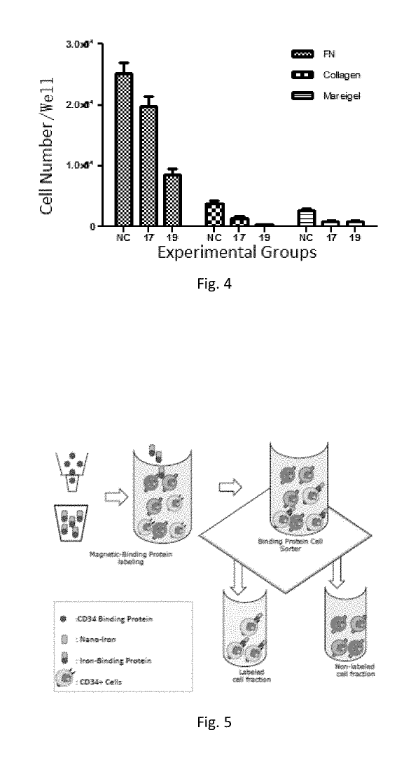

As shown in FIG. 4, incubate K562 cells in 96-hole plates of FN, Collagen I and Mareigel respectively, and set experimental groups and control groups. Add the polypeptides 17 and 19 into incubating holes to set respective experimental groups (final concentration of polypeptide is 2 μg/mL). In the control group (NC in FIG. 4 corresponds to the results of control group), equal amount of polypeptide diluent is added into the incubating holes; remove supernatant and suspended cells 6 hours later, count the adherent cells, the result shows that the culture solution containing the polypeptides 17 and 19 inhibit the adhesion of K562 cells significantly, especially the culture solution containing the polypeptide 19, as shown in FIG. 4.

It can be seen from the experimental results of the example that polypeptides 17 and 19 may be used to develop anti-tumor metastasis drugs for inhibiting the adhesion of CD34 positive tumor cells, and they also can be used to develop the drugs for activating HSC to inhibit the adhesion of CD34 positive HSC/progenitor cell.

### EXAMPLE 5

### Magnetic Separation of CD34 Positive Cells by CD34 Binding Polypeptide

Mix polypeptides 17 and 19 adhering to the surfaces of nano-magnetic beads respectively with K562 cells and peripheral blood cells, and incubate them for 1 hour. Use magnetic sorter to sort, and the sorting efficiency of K562 cells is higher than 95%. Polypeptides 17 and 19 won't bind to CD34 negative cells, so other blood cells cannot be magnetically separated. A schematic diagram of the example is shown in FIG. 5, and the specific operation for magnetic separation of the example can be performed according to conventional magnetic techniques in the art.

It can be seen from the experimental results of the example that polypeptides 17 and 19 can be used to sort CD34 positive cells and remove CD34 negative cells.

### EXAMPLE 6

### Binding Between CD34 Binding Polypeptide and Vascular Endothelial Cells and Ductal Epithelial Cells of Salivary Gland

In the example, immunofluorescence is used to test the binding between polypeptide and ductal epithelial cells and vascular endothelial cells of salivary gland.

Embed the human salivary gland tissues with OCT, and slice them in a thickness of 6 μm at −20° C., fix with acetone for 15 minutes, use PBS to wash them for three times, 5 minutes each time. Incubate them with 2 μg/mL CD34 binding polypeptide (polypeptide 17) at room temperature for 1 hour, use PBS to wash for three times, 10 minutes each time. Drop 20 μL anti-fluorescence quenching agent for mounting, and preserve them being protected from light at 4° C., fluorescence microscope is used to observe and photograph.

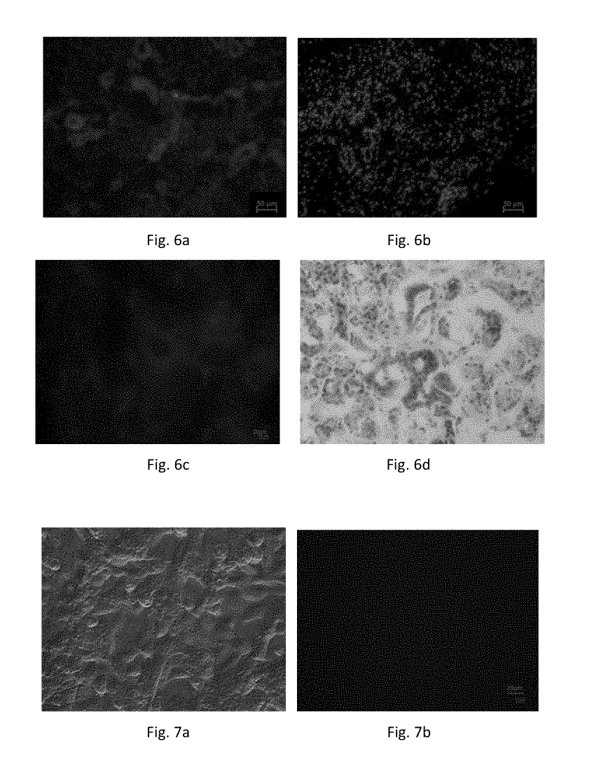

As shown in FIG. 6a, the green fluorescence, distributed around the lumen, is indicative of CD34 binding polypeptides which bind to ductal epithelial cells and vascular endothelial cells of salivary gland tissue. An immunofluorescence test for CD34 expression in salivary gland tissue slices is also performed through CD 34 antibodies (RA), and the result is shown in FIG. 6c, which proves that CD34 expression exists in the ductal epithelial cells and vascular endothelial cells of salivary gland tissue, and the expression area is the same as that of CD34 binding polypeptide (polypeptide 17). H&E staining photo of frozen slice of salivary gland tissues is shown in FIG. 6d, wherein duct cells are in darker staining with eosin.

Further, the same experiments are also performed for the polypeptide 19, the result is similar to the polypeptide 17, not repeated herein.

As can be seen from the experimental results of the example, polypeptides 17 and 19 can be used as markers for ductal epithelial cells and vascular endothelial cells.

### EXAMPLE 7

### Test for the Binding Between CD34 Binding Polypeptide and Human Osteosarcoma Cell Line MNNG/HOS

Add 2 μg/ml polypeptides 17 and 19 into two culture dishes of well-grown MNNG/HOS cells (density of 80%) respectively, incubate them in an incubator at 37° C. for 2 hours, remove the culture solution, use 1 ml PBS to wash them for 3 times, add 5 ml culture solution, and use a fluorescence microscope to observe the binding between polypeptide and MNNG/HOS cells.

As shown in FIGS. 7a to 7b, FIG. 7a shows polypeptide 19 binding MNNG/HOS cells in bright field, and FIG. 7b is a fluorescence microscope photo in the same view. No binding between fluorescent polypeptide and MNNG/HOS cells is observed.

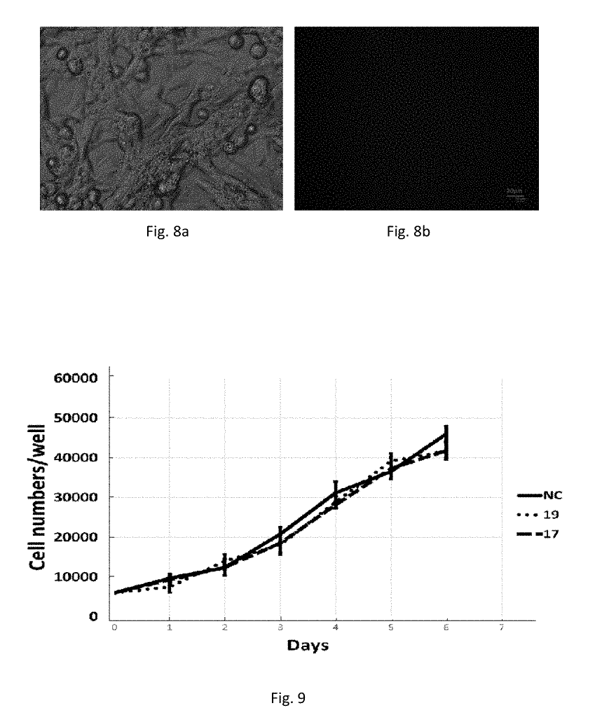

As shown in FIGS. 8a to 8b, FIG. 8a shows polypeptide 17 binding MNNG/HOS cells in bright field, and FIG. 8b is a fluorescence microscope photo in the same view. No binding between fluorescent polypeptide and MNNG/HOS cells is observed.

Osteosarcoma cell line MNNG/HOS does not express CD34 molecule, and the experimental results show the specificity of binding between polypeptides 17, 19 and CD34 molecule.

It can be found in the example that polypeptides 17 and 19 won't bind to human osteosarcoma cell line MNNG/HOS, which shows the binding specificity of CD34 molecules. Similar experimental results are also shown in cell lines such as squamous-cell carcinoma, malignant melanoma and breast cancer, not illustrated one by one herein.

### EXAMPLE 8

### Effect of CD34 Binding Polypeptide on Proliferation of K562 Cells

(1) Inoculate well-grown K562 cells in a 24-hole plate in cell concentration of 6000/ml/hole; set two experimental groups, wherein polypeptides 17 and 19 (polypeptide concentration is 2 μg/ml) are added into culture solutions respectively; set control groups, wherein polypeptide diluent in the same amount of polypeptide is added into the culture solution.

(2) Count once every 24 hours, replenish CD34 binding polypeptide (polypeptide concentration is 2 μg/ml) once every 48 hours, and the counting lasts for 6 days; repeat the experiment three times;

(3) draw cell growth curves, wherein the Y-axis is indicative of mean value of cell density each day, the X-axis is indicative of incubation time.

Results are shown in FIG. 9, the proliferation rate of K562 leukemia cells in the control group (corresponding to the result of NC in the figure) has no significant difference with that of the K562 leukemia cells being added with CD34 binding polypeptides 17 and 19 respectively, which proves that the CD34 binding polypeptide of the present application has little toxic side-effects, and won't affect the characteristics of normal proliferation of K562 cells.

Those described hereinbefore are merely preferred embodiments of the present invention, without any form of restriction for the present invention. Thus contents without departing from the technical solutions of the present invention, any simple changes, equivalent variations and modifications based on the technical essence of the present invention fall into the scope of the present invention.

1. A polypeptide specifically binding to CD34 molecule, comprising:
the polypeptide selected from at least one of polypeptide 17, polypeptide 19 and a combination thereof, wherein an amino acid sequences of the polypeptide 17 is SEQ ID NO:1 and an amino acid sequence of polypeptide 19 is SEQ ID NO: 2.

2. A marker of a CD34 positive expression cell, comprising the polypeptide of claim 1.

3. A CD34 target drug, comprising:
an active components which includes the polypeptide of claim 1.

4. The CD34 target drug according to claim 3, wherein the CD34 target drug further comprises:
a pharmaceutically acceptable carriers and/or excipient.

5. A method for inhibiting adhesion and homing of CD34 positive cells comprising the steps of:
preparing a pharmaceutical formulation comprising a polypeptide of claim 1, and
providing a dosage of the pharmaceutical formulation sufficient to regulate adhesion by inhibiting adhesion and homing of CD34 positive cells via binding the polypeptide of claim 1 to CD34 positive cells.

6. A method for regenerating tissue comprising the steps of:
preparing a pharmaceutical formulations comprising a polypeptide of claim 1; and
adsorbing the polypetide of the pharmaceutical formulation to CD34 positive cells.

7. A composition for identifying, enriching, screening and purifying stem cells comprising of the polypeptide of claim 1.

8. A method for diagnosing leukemia comrprising the steps:
preparing a pharmaceutical formulation comprising a polypeptide of claim 1;
administering the pharmaceutical formulation to a patient suffering from leukemia;
adsorbing the polypeptide of the pharmaceutical formulation to CD34 molecules and;
screening the patient for leukemia by co-immunoprecipitating the CD34 molecules in the leukemia cells with CD34 antibodies.

9. An anti-tumor metastasis drug formulations comprising:
a polypeptide having an amino acid sequence selected from the group consisting of SEQ ID NO:1. SEQ ID NO:2; and
a pharmaceutical carrier, wherein the drug targeting formulation is prepared into a dosage form selected from the group consisting of a capsule, tablet, oral liquid, dispersible table, injectable and freeze-dried powder.

10. A method for detecting and separating CD34 positive cells comprising the steps of:
adhereing a mixture of polypeptides having the amino acid sequence of SEQ ID NO: 1 and SEQ ID NO: 2 to a surface of nano-magnetic beads with a cell having CD34 and peripheral blood cells;
using a magnetic sorter to separate CD34 positive cells bound to the polypeptides from CD34 negative cells that do not bind to the polypeptides.


SEQUENCE LISTING (2 sequences)

SEQ ID NO: 1 - protein, 17 aa, Artificial Sequence
  Non-antibody binding protein capable of binding to CD34
SSFASKKGYPFNRMCAS

SEQ ID NO: 2 - protein, 37 aa, Artificial Sequence
  Non-antibody binding protein capable of binding CD34
SSLGWWGPRRDFPGRRERIYLGIPWLSLLGSGAPSPV